(19) United States
(12) Patent Application Publication
(10) Pub. No.: US 2019/0175017 A1
(43) Pub. Date: Jun. 13, 2019

(54) Maternity Monitoring System and Method

(71) Applicant: Narmadha Kuppuswami, Wheaton, IL (US)

(72) Inventor: Narmadha Kuppuswami, Wheaton, IL (US)

(21) Appl. No.: 16/210,491

(22) Filed: Dec. 5, 2018

Related U.S. Application Data
(63) Continuation-in-part of application No. 15/585,315, filed on May 3, 2017.

(51) Int. Cl. A61B 5/00; A61B 5/021

(52) U.S. Cl. CPC A61B 5/0011; A61B 5/0006; A61B 5/0402; A61B 5/746; A61B 5/7275; A61B 5/021

(57) ABSTRACT

A system and method for monitoring a plurality of parameters of a user that includes receiving the plurality of parameters of the user from a vital signs recording device, identifying at least one abnormal value based on at least one parameter of the plurality of parameters, the at least one parameter being at least one of below a normal range and above a normal range, sending at least one alarm based on the at least one abnormal value to a remote facility, and displaying the at least one alarm continuously on a display device in the remote facility on a condition that the at least one parameter is at least one of below the normal range and above the normal range.

### CROSS-REFERENCE TO RELATED APPLICATION(S)

This application claims priority to and is a continuation-in-part of U.S. Non -provisional application Ser. No. 15/585,315, filed May 3, 2017, to the extent allowed by law and the contents of which are incorporated herein by reference in their entireties.

### TECHNICAL FIELD

This disclosure relates to a system and method for monitoring a plurality of parameters, and more particularly, to a maternity system and method for monitoring a plurality of vital signs of a user and/or patient.

### BACKGROUND

Every day approximately 830 women die from pregnancy or childbirth related complications and preventable causes related to pregnancy and childbirth. Unexpected complications resulting in death or permanent harm to the mother are still unacceptably high around the world. It was estimated that in 2015, roughly 303,000 women died during and/or following pregnancy and childbirth. About 99% of all maternal deaths occur in developing countries. The risk of maternal mortality is highest for adolescent girls under 15 years of age and complications in pregnancy and childbirth is a leading cause of death among adolescent girls in developing countries. Almost all of these deaths occurred in low-resource settings. Maternal mortality is higher in women living in rural areas and among poor communities due to the lack of available resources. However, low resource availability is not the only reason for maternal deaths. Even in the United States, the total number of maternal deaths continues to increase, from 7.2 per 100,000 live births in 1987 to 17.8 per 100,000 live births in 2011. Even more concerning is the fact that for every maternal death, about 100 women will suffer severe maternal morbidity. These women who have gone through severe morbidity may have a similar diagnosis and similar rates of preventable factors as the mothers who did not survive.

Factors that contribute to maternal death in low resources settings most likely are failure to recognize the seriousness of a patient's condition, lack of knowledge, failure to follow recommended practices, lack of or failure to implement policies, protocols and guidelines, and poor organizational arrangements. On the contrary, in developed nations, where skilled obstetrical care is the norm, problems like diagnostic errors, delay in diagnosis, inappropriate or inadequate treatment, and an inability to get the skilled obstetrician at the patient's bedside during acute emergencies are probably contributing to maternal death. Breakdowns in communication and failures of organizational culture and teamwork have consistently ranked among the top three leading contributors to reported maternal and newborn sentinel events. About 75-80% of maternal deaths are preventable. Failure to recognize the warning signs, inadequate care, and missed diagnoses are the major reasons for maternal deaths and “near misses”. Skilled care before, during, and after childbirth can save the lives of women and newborn babies.

Women who ultimately die during childbirth rarely go directly from being in a healthy state to death, but rather go through situations that may lead to severe morbidity when necessary aggressive management is not instituted. Typically, there is a transition period where the patient remains seemingly stable, but abnormal vital signs suggest the potential for impending deterioration. This transition period is the crucial time of intervention in order to prevent death. Intervention in this transition period can save the mother's life, but unfortunately this critical transition period is often under-recognized. These vital signs include, but are not limited to, blood pressure, heart rate, respiratory rate, oxygenation, and temperature. Any vital signs recording device, which can measure these signs, paired with clinical decision support capabilities can assist care providers, especially those in low resource settings and with less experience, in initiating lifesaving treatment.

Hence there is a need for a system and a method that can identify the risk factors early during the initial crucial period, allowing enough time to institute the appropriate care. This method and system should address all of the identified causes of maternal death, should be simple, portable, and cheap and should have the capability to be used in any place regardless of limited resources.

### SUMMARY

This disclosure relates generally to a maternity monitoring system and method. One implementation of the teachings herein is a method for monitoring a plurality of parameters of a user that includes receiving, using a wireless connection, the plurality of parameters of the user from a vital signs recording device; identifying at least one abnormal value based on at least one parameter of the plurality of parameters, the at least one parameter being at least one of below a normal range and above a normal range; sending, using the wireless connection, at least one alarm based on the at least one abnormal value to a remote facility; and displaying the at least one alarm continuously on a display device in the remote facility on a condition that the at least one parameter is at least one of below the normal range and above the normal range.

Another implementation of the teachings herein is a system for monitoring a plurality of parameters of a user that includes a vital signs recording device adapted to identify the plurality of parameters of the user; a wireless communications link adapted to communicate with at least one of the vital signs recording device, a computer at a remote facility, and a mobile electronic device; a software application adapted to monitor the plurality of parameters, identify at least one abnormal value based on at least one parameter of the plurality of parameters, and generate at least one alarm based on the at least one abnormal value; and at least one display device at the remote facility adapted to display the at least one alarm continuously on a condition that the at least one parameter is at least one of below a normal range and above a normal range.

These and other aspects of the present disclosure are disclosed in the following detailed description of the embodiments, the appended claims and the accompanying figures.

### BRIEF DESCRIPTION OF THE DRAWINGS

The various features, advantages, and other uses of the device and method will become more apparent by referring to the following detailed description and drawings, wherein like reference numerals refer to like parts throughout the several views. It is emphasized that, according to common practice, the various features of the drawings are not to-scale. On the contrary, the dimensions of the various features are arbitrarily expanded or reduced for clarity.

FIG. 1 is a perspective view of a first embodiment of a vital signs monitoring and/or recording device, shown worn by a user, in accordance with implementations of this disclosure;

FIG. 2 is an elevation view of the first embodiment of the vital signs monitoring and/or recording device, showing a cuff in an unwrapped position, in accordance with implementations of this disclosure;

FIG. 3 detail front elevation view of a display of the first embodiment of the vital signs monitoring and/or recording device, showing temperature data of the user, in accordance with implementations of this disclosure;

FIG. 4 is a detail front elevation view of the display of the first embodiment of the vital signs monitoring and/or recording device, showing respiration data of the user, in accordance with implementations of this disclosure;

FIG. 5 is a detail front elevation view of the display of the first embodiment of the vital signs monitoring and/or recording device, showing pulse data of the user, in accordance with implementations of this disclosure;

FIG. 6 is a detail front elevation view of the display of the first embodiment of the vital signs monitoring and/or recording device, showing blood pressure data of the user, in accordance with implementations of this disclosure;

FIG. 7 is a flow diagram of a process for monitoring a plurality of physical signs of a user in accordance with implementations of this disclosure;

FIG. 8 is a flow diagram of a first embodiment of a system, showing a workflow of the process for monitoring the plurality of physical signs of a user wirelessly, in accordance with implementations of this disclosure;

FIG. 9 is a diagram of the first embodiment of the system, showing an exemplary implementation of a plurality of physical signs monitored and alerts displayed, in accordance with implementations of this disclosure;

FIG. 10 is a diagram of the first embodiment of the system, showing an exemplary implementation of treatment instructions, in accordance with implementations of this disclosure;

FIG. 11 is a diagram of the first embodiment of the system, showing an exemplary implementation of steps that can be taken at an electronic birth center, in accordance with implementations of this disclosure; and

FIG. 12 is a diagram of the first embodiment of the system, showing an exemplary implementation of alerts and data sent to a hospital, in accordance with implementations of this disclosure.

### DETAILED DESCRIPTION

Every day approximately 830 women die from preventable causes related to pregnancy and childbirth. About 99% of all maternal deaths occur in developing countries. Maternal mortality is higher in women living in rural areas and among poor communities due to the lack of available resources. There are many factors that contribute to maternal death, such as failure to recognize the seriousness of a patient's condition, lack of knowledge, failure to follow recommended practices, lack of or failure to implement policies, protocols and guidelines, and poor organizational arrangements in low resource settings, and diagnostic errors, delay in diagnosis, inappropriate or inadequate treatment, and an inability to get the skilled obstetrician at the patient's bedside during acute emergencies in developed areas. Breakdowns in communication and failures of organizational culture and teamwork have consistently ranked among the top three leading contributors to reported maternal and newborn sentinel events.

There is a need for a system and a method that can identify the risk factors early during the initial crucial period, allowing enough time to institute the appropriate care. This transition period is the crucial time of intervention in order to prevent maternal death. The system and method of the present disclosure can identify the important changes in the vital signs of laboring patients and signals abnormal values, trends, and patterns to alert the care provider to intervene. Any vital signs recording device can be integrated with the system through a wireless connection, which helps avoid measurement and recording errors. The vital signs recording device is simple, portable, and low cost, and has the capability to be used in any location regardless of limited resources. The system can then identify abnormal vital signs and create audible alarms as well as visual alerts and has the ability to generate all reports that are needed for any analysis. In the hospital, a red alert is displayed on the dashboard when the vital signs are critically abnormal and a yellow alert is displayed on the dashboard when the vital signs are mildly abnormal, which helps in early identification to avoid delays. The system also provides instant treatment guidelines for acute obstetric emergencies, from the various organizations, modified to local needs. In addition, the system can also provide possible diagnosis/diagnoses as a pop-up. The system also has a potential for low-level providers, such as nurses and auxiliary nurse midwives, to initiate treatment for any acute obstetrical emergency while waiting for the doctor, which helps address the problem of skilled provider shortage in rural areas. The system functions in real-time and is highly confidential, which provides accountability and accuracy within the tamper-proof system. The system is built with mobile technology and is cloud-based, hence, physicians have the ability to access the patient's record on a mobile electronic device from wherever they are located.

The automatic alert system has the ability to also send instant messages to tertiary care hospitals which in turn can send a transport team to transport the patient safely to their tertiary care center. In this way, the system helps to provide an effective obstetric care (delivery system) to women in hard to reach areas. For example, any vital signs monitoring and/or recording device can be connected to a portable mobile unit that has the system installed through a wireless mechanism. A low-level provider can be given the portable mobile unit and the monitoring device that can be applied to a patient in a remote area. If the system detects that a vital signs value is abnormal, the system can automatically send an instant message to a remote monitoring station, such as an electronic birth center (e-BC), similar to an air traffic control system, because it is a cloud-based system. The vital signs records will be displayed at the e-BC within 30 seconds or less. A skilled provider at the e-BC will be able to assess the patient's vital signs and/or records and, using an internet search, such as Google, Yahoo, and Bing, and/or a navigation system search, such as Google Maps, Maps, and Waze Navigation & Live Traffic, can get the patient to be airlifted and transported to the nearest obstetric care hospital.

Additionally, remote Level 1 obstetrical units can be connected to central monitoring stations, which can be situated in any place or country, such as the United States, the United Kingdom, or anywhere else. For example, the health center could be located in a remote village in Africa, and because this is a cloud-based system, the data from the health center will be visible to the central monitoring station and/or general hospital within 30 seconds or less. Experienced clinicians at the central monitoring station, such as an electronic intensive care unit and/or air traffic control, can provide instructions for acute emergencies and, once the patient is stable, can arrange to airlift the patient to the nearest tertiary center identified by an internet search, such as Google, Yahoo, and Bing, and/or a navigation system search, such as Google Maps, Maps, and Waze Navigation & Live Traffic. This way, skilled obstetric care can be provided to any part of the world without building additional infrastructure.

If there is an acutely ill patient in a peripheral health care center, the skilled provider at the tertiary care center can guide them and/or provide treatment instructions to stabilize and transport the patient once the patient is stable. Prior teaching and/or training of low level providers about how to stabilize an unstable patient will help the stabilization process as well. If both centers are connected electronically, the medical records will be available from the peripheral health care center to the tertiary center within 30 seconds if not sooner. If the physician at the peripheral health care center feels that he/she cannot address the patient's complicated issue, then that particular patient can be directly transported from the peripheral health care center to a tertiary care center based on the physician's evaluation, thereby avoiding any delay.

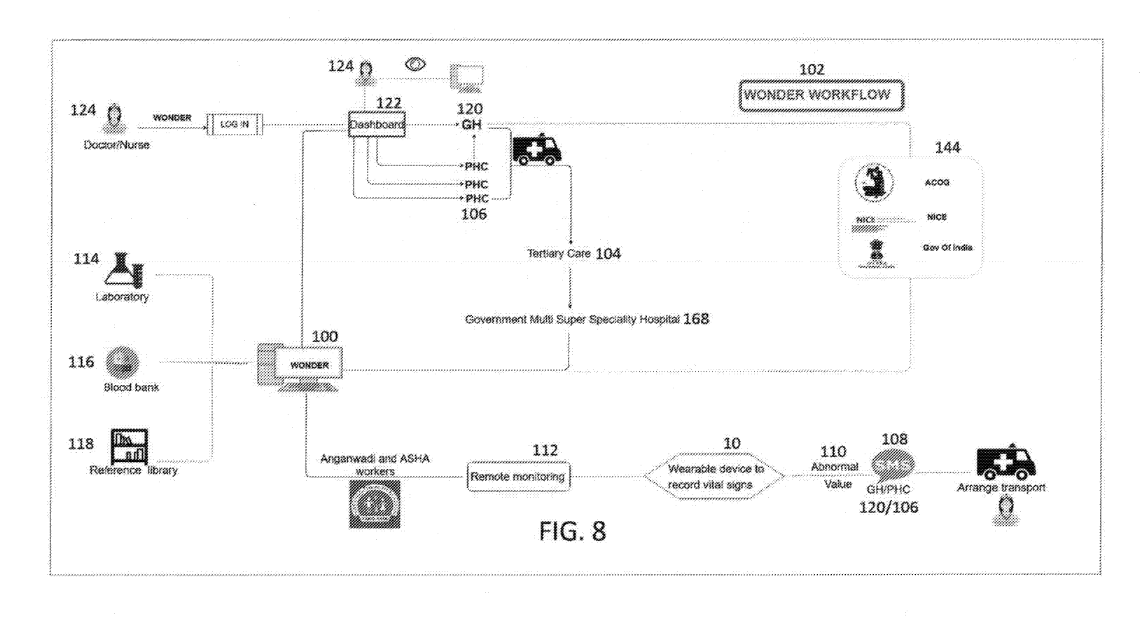

Referring to FIG. 8, an exemplary implementation of a workflow 102 of a first embodiment of an electronic health record system 100 is shown. The electronic health record system 100 provides women's obstetrical neonatal death evaluation and reduction services to areas around the world and is highly confidential and tamper-proof, such that it complies with electronic medical records regulations and can be used anywhere in the world. The electronic health record system 100, in this exemplary implementation, is based on a maternal obstetric early warning score system (not shown) and a newborn early warning system (not shown), an alert system that is based on set criteria to avoid delays, instant pop ups of up to date treatment guidelines that can be modified based on local needs, and cloud-based two-way communications between a tertiary care center 104 and a peripheral low resources hospital 106 forming one unit of an obstetric care delivery system (not shown). The electronic health record system 100 can be integrated by any number of vital signs recording devices, such as a monitoring device described in Applicant's co-pending U.S. Non-provisional patent application Ser. No. 15/585,315, which is incorporated herein by reference in its entirety.

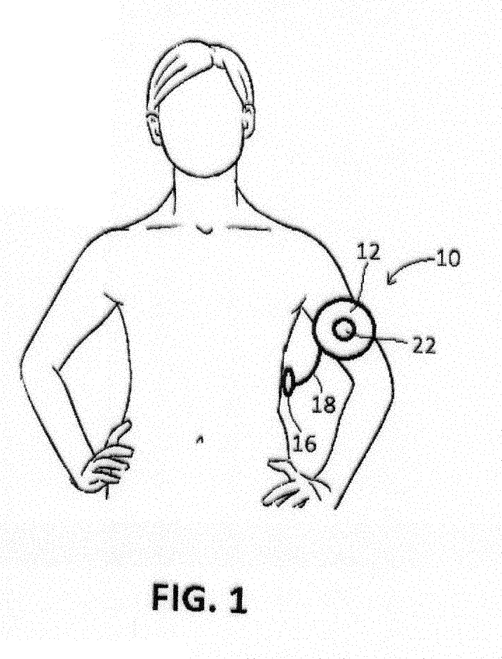

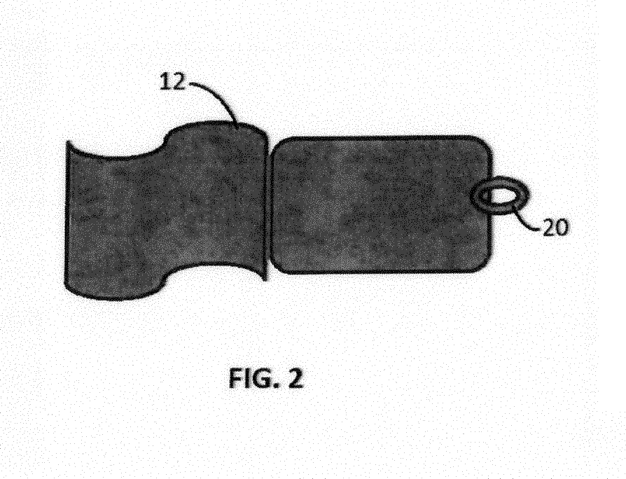

Referring to FIG. 1, a first embodiment of a vital signs monitoring and/or recording device 10 is shown on a patient. The monitoring device 10 comprises an inflatable cuff 12 (FIGS. 1 and 2) and two sensors 14 (not shown), 16 (FIG. 1). Sensor 14 is disposed on the inflatable cuff 12 such that when the cuff 12 is wrapped around the upper arm of the patient, sensor 14 is placed over the inner aspect of the upper arm against a major artery, such as the brachial artery. Sensor 14, when the cuff 12 is inflated and deflated, can check the patient's blood pressure and pulse. The cuff 12 comes in various sizes to accommodate the patient's arm size and includes hook and look fasteners (not shown) allowing the cuff 12 to be wrapped and secured around the arm of the patient. Sensor 16 is applied to the chest wall of the patient, just below the axilla, and is connected to sensor 14 via a loose cord 18 that will not limit the patient's arm movement. Sensor 16 is configured to check the patient's temperature and count the patient's chest wall movements with respiration, thereby monitoring the patient's respiratory rate.

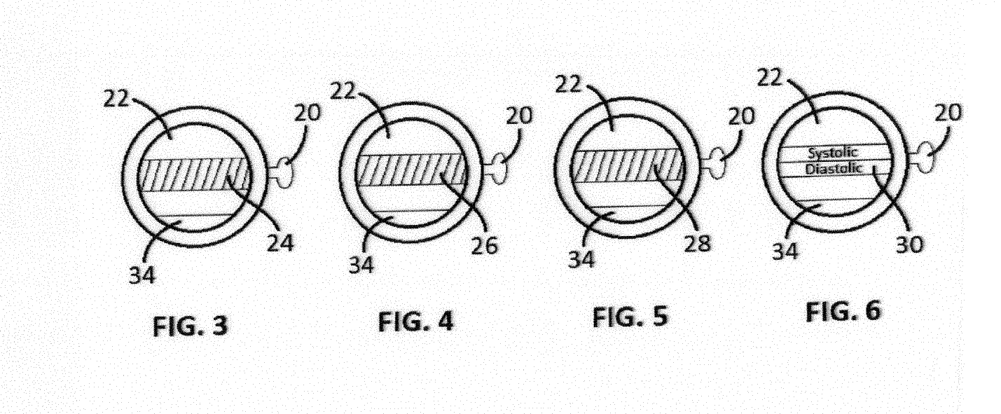

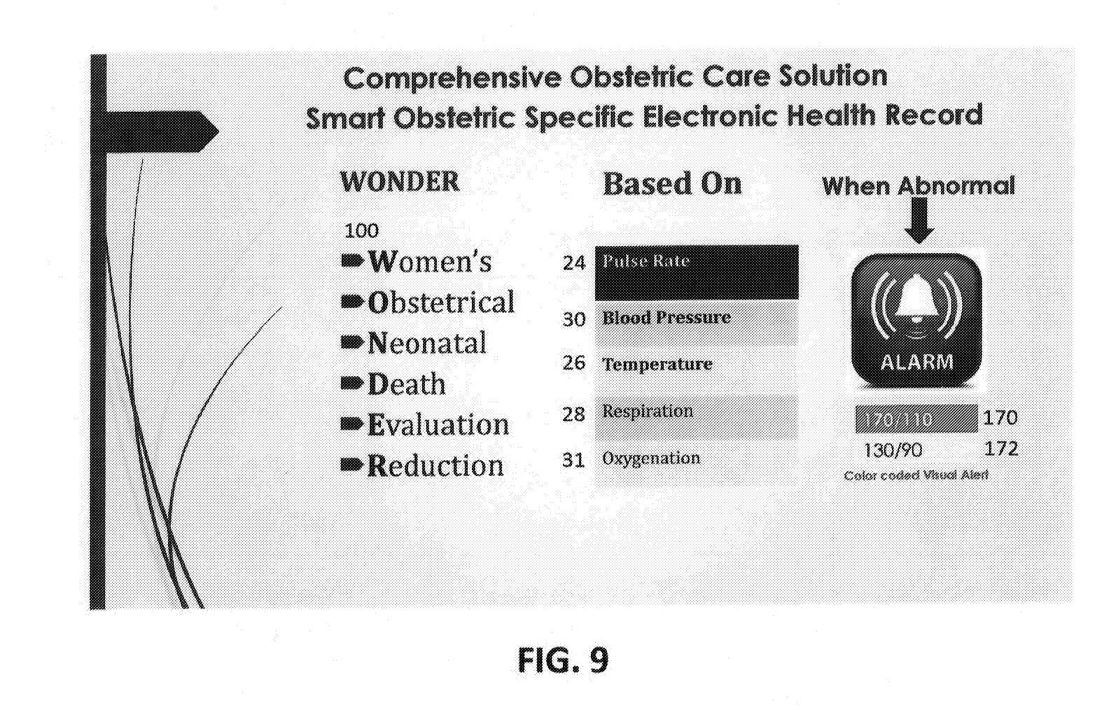

In this implementation, the monitoring device 10 can also comprise a dial 20, shown in FIGS. 2-6, that can be turned to display a plurality of parameters on a display 22, shown in FIGS. 1 and 3-6. The rotatable dial 20 allows the user to select between parameters and additional programming options. The plurality of parameters in this exemplary implementation includes pulse, temperature, respiratory rate, oxygenation, and blood pressure. In this implementation, the dial 20 has four settings that each display a vital sign parameter of the plurality of parameters in a graphic or numerical value format and a date and time display 34, shown in FIGS. 3-6, on the display 22 and one additional setting that allows for additional device programming. A first setting, as shown in FIG. 3, displays the patient's pulse rate 24 in beats per minute and can also be displayed in graph form on the display 22, allowing a medical professional to easily identify an upward or downward trend when displayed as a graph as time over value. If the patient's pulse rate 24 is within normal range, the pulse 24 will display the pulse rate 24 in the normal range of display 22. A second setting, as shown in FIG. 4, displays the patient's temperature 26 in Centigrade or Fahrenheit and can also be displayed in graph form on the display 22, allowing the medical professional to easily identify an upward or downward trend when displayed as a graph as time over value. If the patient's temperature 26 is within normal range, the temperature 26 will display the temperature 26 in the normal range of display 22. A third setting, as shown in FIG. 5, displays the patient's respiratory rate 28 per minute and can also be displayed in graph form on the display 22, allowing the medical professional to easily identify an upward or downward trend when displayed as a graph as time over value. If the patient's respiratory rate 28 is within normal range, the respiratory rate 28 will display the respiratory rate 28 in the normal range of display 22. A fourth setting, as shown in FIG. 6, displays the patient's blood pressure 30 as systolic pressure over diastolic pressure on the display 22, measured in millimeters of mercury (mmHg), above the surrounding atmospheric pressure (considered to be zero for convenience). The systolic and diastolic pressures can also be displayed in graph form on the display 22, allowing the medical professional to easily identify an upward or downward trend when displayed as a graph as time over value. If the patient's blood pressure 30 is within normal range, the blood pressure 30 will display the blood pressure 30 in the normal range of display 22. A fifth setting displays the patient's oxygenation 31 (FIG. 9), which is the fraction of oxygen-saturated hemoglobin relative to total hemoglobin (unsaturated+saturated) in the blood. The oxygenation 31 can also be displayed in graph form on the display 22, allowing the medical professional to easily identify an upward or downward trend when displayed as a graph as time over value. If the patient's oxygenation 31 is within normal range, the oxygenation 31 will display in the normal range of display 22.

The monitoring device 10 includes a built-in battery and a software application that can be programmed to check various vital signs, or plurality of parameters, at predetermined or customized time intervals or as often as needed. The monitoring device 10 also comprises a built-in automatic alert system for detecting abnormal values based on given parameters. Alert parameters for each of the vital sign plurality of parameters, for the first setting 24, the second setting 26, the third setting 28, the fourth setting 30, and the fifth setting 31 will be set at upper and lower normal ranges. Any value that is abnormal 110, either above an upper normal range or below a lower normal range, will automatically be rechecked in a configurable or predetermined amount of time, such as ten minutes. Once rechecked, the monitoring device 10 will set off an alarm, such as a beep, if any parameter remains outside of the normal range. In another implementation, any value that is abnormal 110, either above an upper normal range or below a lower normal range, will generate an alarm within 30 seconds or less, in this exemplary implementation, such as an audible alarm, a visual alarm, and/or an instant message 108 to the peripheral health care center 106 and the alarm will continue with each recording of the patient's vital signs until the patient's vital signs are stable, as shown in FIG. 9. The visual alarm in this exemplary implementation is displayed as a red alert or a yellow alert. Any of the patient's vital sign parameters (pulse, temperature, blood pressure, respiratory rate, oxygenation, etc.) can be checked at any interval and the monitoring device 10 can be programmed to monitor additional user parameters using the additional device programming setting 32 (not shown) on dial 20.

The software application can also include an automatic messaging system that provides the patient's parameters and/or history to any designated center via a variety of wireless communications links. The wireless data link can be, for example, a wireless local area network (WLAN), wireless metropolitan area network (WMAN), wireless wide area network (WWAN), a private wireless system, a cellular telephone network or any other means of transferring data and/or messages from the monitoring device 10 to, in this example, a remote designated center. The monitoring device 10 can also connect via wireless access to the smart electronic obstetrical health record system 100 and transmit and/or store the patient's parameters and/or history, providing the health record system 100 with remote monitoring 112 of the patient's vital signs, medical records, and/or history.

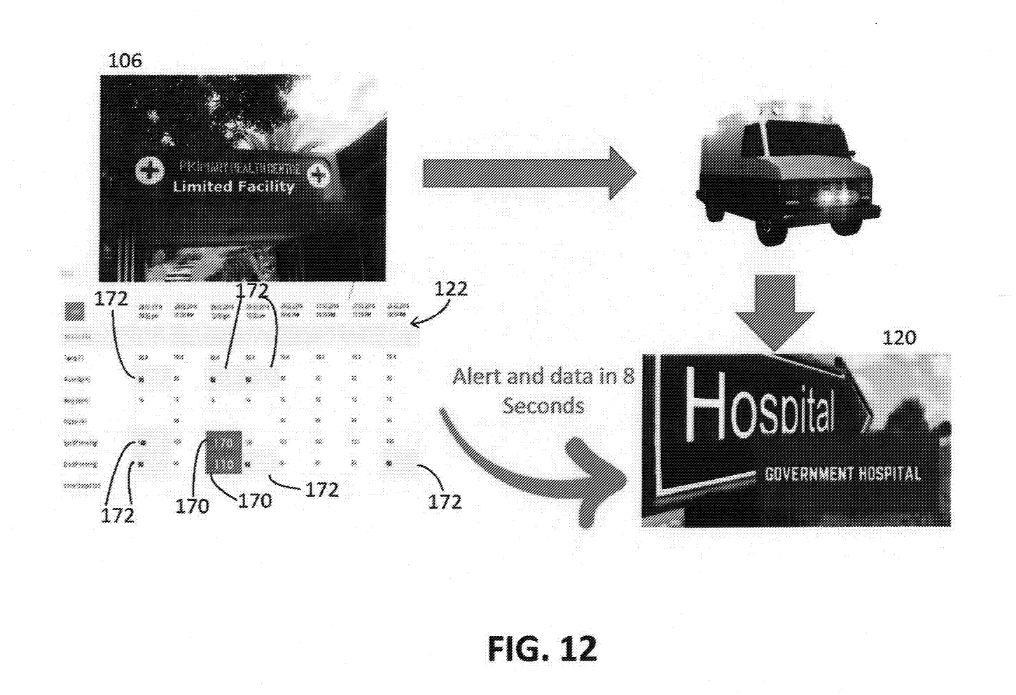

The health record system 100 can identify abnormal vital signs, create audible alarms, visual alarms, and/or instant messages to provide automatic alerts, and generate any reports that are needed for analysis of the patient's current condition. The health record system 100 also has access to the patient's laboratory results 114 and blood bank results 116 and to a reference library 118. Based on the patient's vital signs, medical records, laboratory results 114, blood bank results 116, history, and/or reference library 118, the health record system 100 can generate and send the reports and/or alerts to general hospital 120 and/or peripheral low resources hospital 106. The health record system 100 can then display any of the patient's vital signs, medical records, laboratory results 114, blood bank results 116, history, reference library 118, reports and/or alerts on a dashboard 122 in the general hospital 120 and/or peripheral low resources hospitals 106. In the hospital 120, 106, in this exemplary implementation, a red alert 170 (FIGS. 9 and 12) is displayed on the dashboard 122 when the patient's vital signs are critically abnormal and a yellow alert 172 (FIGS. 9 and 12) is displayed on the dashboard 122 when the patient's vital signs are mildly abnormal (FIG. 12). In the general hospital 120, patients with abnormal yellow alerts 172 and/or red alerts 170 will be displayed on the dashboard 122 at an easily visible spot along with the patients' bed numbers, allowing the providers to quickly identify which patient needs their attention right away and allowing the physician's valuable time to be focused on the sickest patient who needs immediate care to save her life first. Additionally, a doctor, nurse, or medical professional 124 can log into the health record system 100 and the dashboard 122 of the hospital 120, 106 to access the patient's vital signs, medical records, laboratory results 114, blood bank results 116, history, reference library 118, reports and/or alerts.

For example, at the tertiary care center 104, if the patient's lab results show a critically low hemoglobin level, the system 100 will send an alert to the blood bank to alert them to get their resources ready for a possible massive transfusion. In the Level I center, since the system 100 has integrated lab results, if the lab results show a low hemoglobin level indicating severe anemia, the system 100 will send an automatic SMS message to the patient explaining that she has severe anemia and needs to get to the hospital for treatment. At the same time, the system 100 sends an alert to the provider 124 regarding the patient with the low hemoglobin level so the provider 124 can make sure that a village health nurse or other medical professional will get involved in helping the patient with transportation arrangements to bring the patient to the hospital for treatment.

The monitoring device 10 can be used as a stand-alone system with the built-in alert/alarm system and automatic messaging system. The monitoring device 10 can also be used with a smartphone and/or a computer, where the device 10 can instantly connect to the cloud -based software system 100 and generate instant pop-ups with diagnoses and up-to-date and detailed management guidelines. The cloud-based software system 100 can be uploaded to or integrated with the monitoring device 10, which helps the provider in patient management.

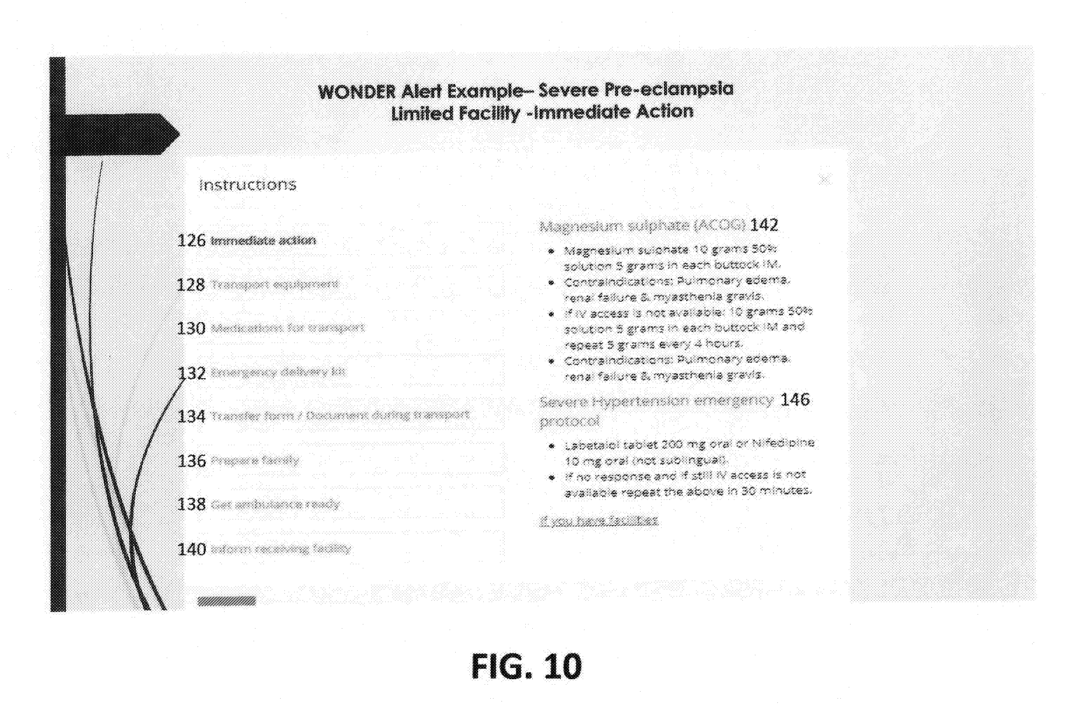

The cloud-based health care system 100 can also provide real-time instant treatment guidelines for acute obstetric emergencies, from various organizations 144, modified to local needs to allow low-level providers to initiate treatment for any acute obstetric emergency while waiting for a doctor. Treatment guidelines and/or instructions, in this exemplary implementation, include, but are not limited to, immediate action 126, transport equipment 128, medications for transport 130, emergency delivery kit 132, transfer form/document during transport 134, prepare family 136, get ambulance ready 138, and inform receiving facility 140, as shown in FIG. 10. These treatment guidelines and/or instructions, in this embodiment, are pop-ups that provide instructions to nurses, doctors, emergency medical technician, and medical professionals on what to do, what labs to perform, what medications to administer, etc., in real-time so there is no delay in treating the patient. The exemplary implementation shown in FIG. 10 shows the immediate action 126 instructions for a patient for severe pre-eclampsia in a limited facility, which include magnesium sulphate treatment 142, per the American College of Obstetricians and Gynecologists (ACOG) and/or the National Institute for Health and Care Excellence (NICE) guidelines, and severe hypertension emergency protocol treatment 146. The system 100 tracks the time the medication and/or treatment was started, monitors how long it takes for the patient's vital signs to stabilize, which in turn monitors the effectiveness of the medication and/or treatment, and if the patient is not responding, modifies the medication, dose, and/or treatment instructions, which are evidence based.

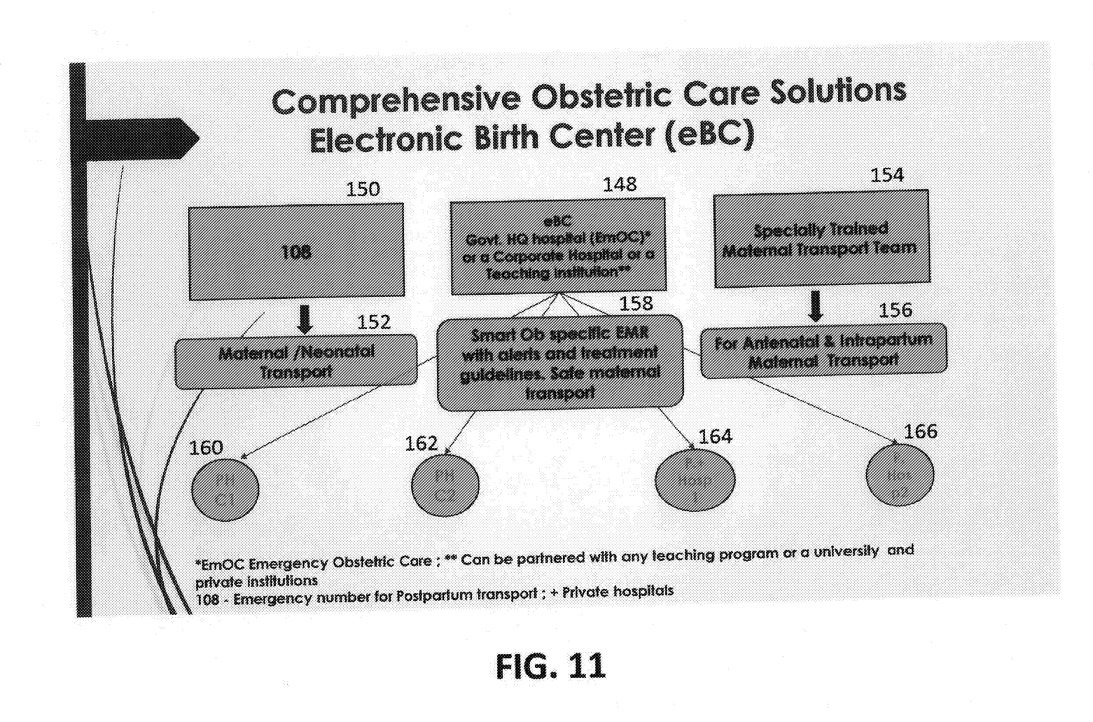

Referring to FIG. 11, an exemplary implementation of the cloud-based health care system 100 is shown implemented in a comprehensive obstetric care solutions electronic birth center (eBC) 148, which can include a government hospital providing emergency obstetric care, a corporate hospital providing emergency obstetric care, and/or a teaching institution 168 (FIG. 8) providing emergency obstetric care that can be partnered with any teaching program or university and/or private institutions. Dependent on the patient's vital signs, medical records, laboratory results 114, blood bank results 116, history, reference library 118, reports and/or alerts, which can be received by the government hospital providing emergency obstetric care, the corporate hospital providing emergency obstetric care, and/or the teaching institution 168 providing emergency obstetric care in less than thirty seconds, or eight second or less, (FIG. 12), the eBC 148 can call an emergency number 150 for postpartum transport, maternal transport, and/or neonatal transport 152 of the patient, the eBC 148 can call a specially trained maternal transport team 154 for antenatal and/or intrapartum maternal transport 156 of the patient, and/or the eBC can additionally provide smart obstetric specific emergency medical response with alerts and treatment guidelines, as described above, and then arrange for safe maternal transport 158 once the patient is stable. The postpartum transport, maternal transport, and/or neonatal transport 152, the antenatal and/or intrapartum maternal transport 156, and/or the safe maternal transport 158 of the patient can be to any number of peripheral health care centers 106, such as peripheral health care center one 160 and peripheral health care center two 162, or to any private hospital, such as private hospital one 164 and private hospital two 166. Once the patient is stable, the health care system 100 can review the data to identify what the patient's problem was and the events that occurred to institute effective responses. The health care system 100 can also determine whether the patient's problem was associated with a locality or region or whether the patient's problem was related to sweeping changes across the board.

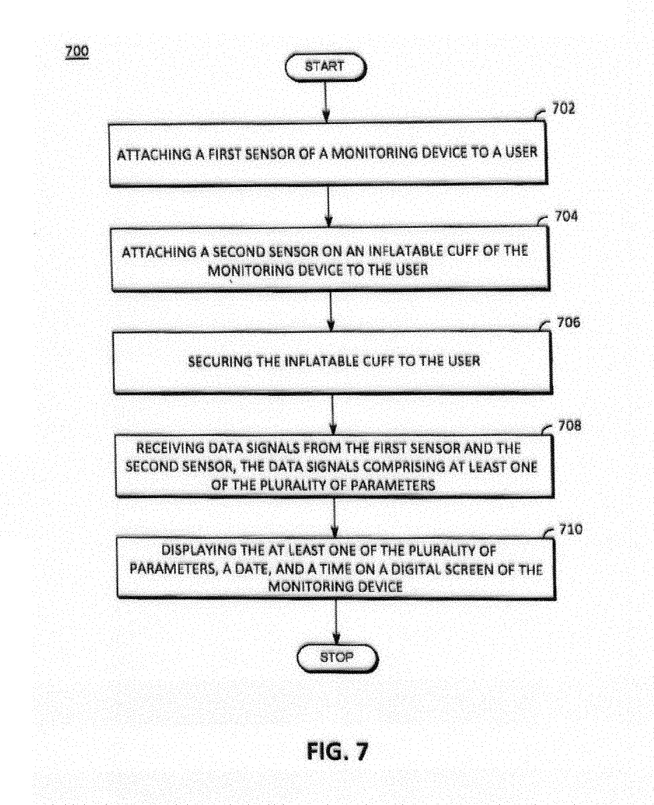

FIG. 7 is a flow diagram showing a process 700 for monitoring a plurality of parameters of a user in accordance with an implementation of this disclosure. The monitoring device 10 is placed on the patient by attaching the sensor 16 of a monitoring device 10 to the chest wall below an axilla of the patient 702. Sensor 14 is then placed over the inner aspect of the upper arm of the patient over a major artery of the patient 704. The inflatable cuff 12 is secured around the upper arm of the patient 706. The monitoring device 10 receives data signals from the sensor 16 and the sensor 14, where the data signals comprises at least one of the plurality of parameters 708. The monitoring device 10 then displays the at least one of the plurality of parameters, a date, and a time 710 on the display 22. The monitoring device 10 then automatically records and stores the patient's vital signs within monitoring device 10, sends alerts when values are abnormal, and sends automatic messages to the peripheral health care center 106 and to the health care system 100. The health care system 100 provides pop-ups with up-to-date treatment guidelines when connected through a computer or mobile electronic device to the electronic obstetrical health record system 100 through the wireless connection. The data recorded and stored within the monitoring device 10 and the health care system 100 is time sensitive, accurate, safe, and secure, complying with the requirements of the Health Insurance Portability and Accountability Act of 1996 (HIPPA). The monitoring device 10 and the health care system 100 can send instant messages to a designated center with emergency care facilities, which can be crucial in cases where the patient is located in a low resources center or location.

As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, “X includes at least one of A and B” is intended to mean any of the natural inclusive permutations. That is, if X includes A; X includes B; or X includes both A and B, then “X includes at least one of A and B” is satisfied under any of the foregoing instances. The articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment, aspect or implementation unless described as such.

While the present disclosure has been described in connection with certain embodiments and measurements, it is to be understood that the present disclosure is not to be limited to the disclosed embodiments and measurements but, on the contrary, is intended to cover various modifications and equivalent arrangements included within the scope of the appended claims, which scope is to be accorded the broadest interpretation so as to encompass all such modifications and equivalent structures as is permitted under the law.

1. A method for monitoring a plurality of parameters of a user comprising:
receiving, using a wireless connection, the plurality of parameters of the user from a vital signs recording device;
identifying at least one abnormal value based on at least one parameter of the plurality of parameters, the at least one parameter being at least one of below a normal range and above a normal range;
sending, using the wireless connection, at least one alarm based on the at least one abnormal value to a remote facility; and
displaying the at least one alarm continuously on a display device in the remote facility on a condition that the at least one parameter is at least one of below the normal range and above the normal range.

2. The method of claim 1, wherein the at least one alarm is at least one of a visual alarm and an audible alarm.

3. The method of claim 1, wherein the at least one alarm is one of red on a condition that the at least one abnormal value is critically abnormal and yellow on a condition that the at least one abnormal value is mildly abnormal.

4. The method of claim 1, further comprising:
sending, using the wireless connection, a message to the remote facility based on the at least one abnormal value.

5. The method of claim 1, further comprising:
generating a report based on at least one of the plurality of parameters of the user, medical records of the user, laboratory results of the user, blood bank results of the user, medical history of the user, and reference library; and
sending, using the wireless connection, at least one of the report, the plurality of parameters of the user, the medical records of the user, the laboratory results of the user, the blood bank results of the user, the medical history of the user, and the reference library to the remote facility.

6. The method of claim 1, wherein the remote facility is at least one of a tertiary health care center, a peripheral health care center, and a general hospital.

7. The method of claim 1, further comprising:
removing the at least one alarm from the display device in the remote facility on a condition that each of the parameters of the plurality of parameters are within the normal range.

8. The method of claim 1, further comprising:
providing, using the wireless connection, treatment instructions to at least one of the user and a medical professional in real time, the treatment instructions comprising at least one of immediate action instructions, transport equipment instructions, medications for transport instructions, emergency delivery kit instructions, transfer form, prepare family instructions, get ambulance ready instructions, and inform receiving facility instructions.

9. The method of claim 8, further comprising:
providing a first immediate action instruction comprising a first dose of at least one medication to administer to the user, the first immediate action instruction based on the at least one abnormal value;
identifying a time the at least one medication was initially administered to the user;
monitoring the plurality of parameters of the user; and
providing a second immediate action instruction on a condition that the at least one of the at least one abnormal value and at least one parameter of the plurality of parameters are at least one of below the normal range and above the normal range, the second immediate action instruction comprising at least one of a second dose of the at least one medication and a dose of at least one second medication.

10. The method of claim 1, further comprising:
sending, using the wireless connection, an instant message to the user, the instant message comprising an explanation of the user's condition and instructions to go to the hospital.

11. The method of claim 1, wherein the plurality of parameters comprises at least two of temperature, pulse, respiratory rate, blood pressure, and oxygenation.

12. A system for monitoring a plurality of parameters of a user comprising:
a vital signs recording device adapted to identify the plurality of parameters of the user;
a wireless communications link adapted to communicate with at least one of the vital signs recording device, a computer at a remote facility, and a mobile electronic device;
a software application adapted to monitor the plurality of parameters, identify at least one abnormal value based on at least one parameter of the plurality of parameters, and generate at least one alarm based on the at least one abnormal value; and
at least one display device at the remote facility adapted to display the at least one alarm continuously on a condition that the at least one parameter is at least one of below a normal range and above a normal range.

13. The system of claim 12, wherein the at least one alarm is at least one of a visual alarm and an audible alarm.

14. The system of claim 12, wherein the at least one alarm is one of red on a condition that the at least one abnormal value is critically abnormal and yellow on a condition that the at least one abnormal value is mildly abnormal.

15. The system of claim 12, the software application further configured to:
send a message to the remote facility based on the at least one abnormal value.

16. The system of claim 12, the software application further configured to:
generate a report based on at least one of the plurality of parameters of the user, medical records of the user, laboratory results of the user, blood bank results of the user, medical history of the user, and reference library; and
send at least one of the report, the plurality of parameters of the user, medical records of the user, laboratory results of the user, blood bank results of the user, medical history of the user, and reference library to the remote facility.

17. The system of claim 12, wherein the remote facility is at least one of a tertiary health care center, a peripheral health care center, and a general hospital.

18. The system of claim 12, the software application further configured to:
remove the at least one alarm from the display device at the remote facility on a condition that each of the parameters of the plurality of parameters are within normal range.

19. The system of claim 12, the software application further configured to:
provide treatment instructions to at least one of the user and a medical professional in real time, the treatment instructions comprising at least one of immediate action instructions, transport equipment instructions, medications for transport instructions, emergency delivery kit instructions, transfer form, prepare family instructions, get ambulance ready instructions, and inform receiving facility instructions.

20. The system of claim 19, the software application further configured to:
provide a first immediate action instruction comprising a first dose of at least one medication to administer to the user, the first immediate action instruction based on the at least one abnormal value;
identify a time the at least one medication was initially administered to the user;
monitor the plurality of parameters of the user; and
provide a second immediate action instruction on a condition that the at least one of the at least one abnormal value and at least one parameter of the plurality of parameters are at least one of below the normal range and above the normal range, the second immediate action instruction comprising at least one of a second dose of the at least one medication and a dose of at least one second medication.

21. The system of claim 12, the software application further configured to:
send an instant message to the user, the instant message comprising an explanation of the user's condition and instructions to go to the hospital.

22. The system of claim 12, wherein the plurality of parameters comprises at least two of temperature, pulse, respiratory rate, blood pressure, and oxygenation.
